(19) United States
(12) Patent Application Publication
(10) Pub. No.: US 2007/0255244 A1
(43) Pub. Date: Nov. 1, 2007

(54) Absorbent article

(75) Inventors: Ken Olsson, Vastra Frolunda (SE); Monica Hager, Landvetter (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 11/819,736

(22) Filed: Jun. 28, 2007

Related U.S. Application Data
(63) Continuation of application No. PCT/SE04/02028, filed on Dec. 29, 2004.

(51) Int. Cl. A61F 13/15

(52) U.S. Cl. 604/385.14; 604/385.01; 604/378

(57) ABSTRACT

An absorbent article (1, 201) has a topsheet (2), a backsheet (3) and at least two absorbent cores (6, 7, 206, 207) placed therebetween. The article (1, 201) is separable, wherein, after separation, the article (1, 201) forms two separate articles (301, 401), at least one of which is primarily intended for urine absorption. This separable article gives great flexibility to the user who can choose whether they will have a urine- and faeces-receiving article, or only a urine-absorbing article by separating the article to two smaller articles.

### CROSS-REFERENCE TO RELATED APPLICATIONS

The present application is a continuation of PCT/SE2004/002028, filed on Dec. 29, 2004, and which designates the U.S. The entire contents of PCT/SE2004/002028 are incorporated herein by reference.

### TECHNICAL FIELD

The invention relates to an absorbent article having at least two absorbent cores.

### BACKGROUND

Urine incontinence has long been a problem for men and women, and great resources have been applied to the development of different absorbent articles for this purpose. A series of different articles, such as pant-diapers, belt-diapers or e.g. simple panty-liners are frequently used by people having lighter or more severe incontinence. In addition to normal urine incontinence, people also exist who suffer from faeces incontinence. Furthermore, there are those who suffer from a combination of faeces and urine incontinence.

Those people who have both urine and faeces incontinence are understandably subject to extra prejudices in society, which can make it embarrassing and difficult as they have to deal with their problems daily. Furthermore, they have purely practical problems, such as often having to buy a variety of different products to satisfy their requirements. This problem can be especially evident for incontinent men, who may need to use a urine incontinence shield and also a faeces shield.

As the normal incontinence diapers of today are often large and bulky, their use is often avoided by those who have a lesser degree of urine incontinence. If one has a lesser degree of urine incontinence, and additionally is faeces incontinent, there is in principle no single product which can satisfy the requirements which one might possibly have. If one is also physically active at work, meets many people or is often seen in public, it becomes understandably even more difficult to be incontinent. A flexible lifestyle in today's society requires flexible solutions. This requirement is specifically found for people who are urine incontinent and who sometimes unwillingly secrete faeces.

Prior to the present invention, there existed a need for a product solution which satisfies the requirements which can arise for those people which have urine incontinence and occasional faeces incontinence. There was a need for a flexible solution for those people who have urine incontinence and faeces incontinence which minimizes the need for many products to be used. Today, these people, and specifically men with these problems, have great problems in finding an absorbent article which meets their requirements.

EP 1 214 920 A2 describes a panty-liner which can be divided so that a better fit can be achieved. Specifically, the panty-liner is divided so that two panty-liners for thong underwear are formed. The absorbent cores in the panty-liner are welded with e.g. heat-welding through the absorbent core. The welds contribute to maintaining integrity of the absorbent core upon separation. However, if the absorbent core comprises a number of layers of e.g. different material, it can be very difficult to weld through the whole thickness of the absorbent core, as the various materials can have different melting points. Articles of this type therefore show a variety of disadvantages. A common disadvantage is that component leakage of the absorbent material, due to welds in the panty liner breaking upon separation of the panty-liner. Another disadvantage is that liquid leakage can occur when the welding through the absorbent core is not sufficient to provide liquid impermeability. There is therefore a need for a solution which does not give rise to component leakage or liquid leakage.

### OBJECTS AND SUMMARY

An absorbent article has been achieved which significantly minimizes the problem with previously-known articles. The article is primarily distinguished by the article having at least two absorbent cores which are arranged at a distance from each other seen in the longitudinal direction, and are designed to be separable from each other, wherein, after separation, the article forms two separate articles, each of which has at least one absorbent core. Upon separation, the article can form two articles intended for receiving faeces. Alternatively, upon separation, the article can form two articles, wherein one is intended for urine absorption and the other intended for receiving faeces. A preferred embodiment is that in which, upon separation, the article forms two articles intended for urine absorption. The separable article provides great flexibility to the user who can choose whether they will have a urine- and faeces-receiving article or two urine-absorbing articles by separating the article to two smaller articles.

By “separable articles”, it is meant that the articles which are formed after separation of the article are each capable of absorbing urine and/or function for receiving faeces. It should be noted that all articles may be cut or pulled apart, although this is not considered to fall within the definition of being “separable” or exhibit means or indication for separating. The article must therefore be designed to be separated.

In one embodiment, the absorbent article is—before separation—intended to handle urine and faeces. This implies that the article can be used for taking care of both urine and faeces, but after separation is only intended to be used for control of urine. Preferably, after separation, the article has two essentially identical absorbent articles intended to absorb urine. The invention gives especially incontinent men the option of choosing whether they will use the article for control of urine and faeces, or only control of urine. Further, two urine absorbent articles are produced, which provides the user with an article which is value for money and flexible.

According to one embodiment, said at least two absorbent cores are separated from each other via an area which is free from absorbent material of the absorbent core. According to one aspect of the invention, the topsheet and the backsheet are joined to each other, so that there is no direct liquid communication between the at least two absorbent cores separated from each other. The area free from absorbent core material suitably has a length seen in the longitudinal direction (y) of 10-60 mm, preferably 15-50 mm, most preferably 15-40 mm.

To simplify the separation of the article into two articles, in one embodiment, there is at least one indication for separation, wherein upon separation along said indication, the article forms two individual separate articles, intended e.g. for urine absorption. By “indication for separation”, is meant an indication to the user as to where the user should separate the article to obtain the two smaller articles. The indication for separation may comprise a folding indication, a color marking, e.g. a dashed and/or a continuous colored line, a figure showing e.g. a pair of scissors or the like. Combinations of the above-mentioned indications are also possible.

In one embodiment, to simplify separation of the article into two articles, there is at least one means for separation. By “means for separation” is meant a mechanism of some sort which makes the separation easier in a purely physical sense. Examples of means for separation are perforations, heat-treated lines which weaken the material, threads with which one can tear off the article, or other similar means. Combinations of the above-mentioned means are also possible.

Combinations of indications and means for separation are also possible. An example of this is perforations and a colored dashed line. The colored line made is clear to the user where they can separate the article so that two articles are e.g. for urine absorption. Perforations allow the article to be easily separated by hand by the user simply pulling the article apart along the perforations.

Another example of a combination is a folding indication and a perforation. Preferably the perforation and the folding indication coincide. The folding indication is preferably placed on the article's transverse center-line. It is particularly advantageous that, upon packing, the absorbent article can be folded along the transverse center-line so that space and packaging material can be saved. Perforations then make separation of the article easier. The above-named combinations should only be considered as examples. There may be a series of other combinations within the scope of the invention.

In one embodiment, the article's length before separation is at least 400 mm, preferably at least 520 mm. By having such a long article, vital genitalia are covered and leakage of faeces or urine is minimized. A good fit is obtained for users of the male or female sex. In one embodiment, the length of the described article after separation is at least 100 mm, preferably at least 150 mm, more preferably at least 200 mm and most preferably 250 mm, as measured along the article's longitudinal center-line according to FIG. 1.

In one embodiment, the indications for separation are located in the area which is free from absorbent core between the two absorbent cores which are separated from each other. This leads to the absorbent cores in each of the articles which are formed are still completely surrounded by topsheet and backsheet. This effectively minimizes liquid leakage and possible waste of fibers, superabsorbents or other components from the absorbent article—in the following called “component leakage”. In that the indication or the means for separation are placed between the two absorbent cores which are separated from each other, a higher certainty against component leakage is obtained than, for example, welding through the absorbent core and around the separation indications.

The area which is free from absorbent core not only gives a good and clear separation area, but also a good fit in the user's crotch area. By the user's “crotch area”, is meant the general area between the genitals and anus, i.e. between the legs.

One embodiment of the article is suitable for male users. After separation, the article hereby becomes two articles which are especially formed to absorb urine of incontinent males. Before separation, the two separate absorbent cores give good protection against faeces and urine incontinence, and good fit between the user's legs, i.e. in the crotch area. Before separation, the two separate absorbent cores can be formed for use only for urine incontinence. The only function which the separation function has here is to give the user a cheaper alternative. After separation, two shields for urine absorption are produced. The separate absorbent cores are preferably substantially triangle-shaped. By having a substantially triangle-shaped absorbent core, or by the absorbent core after separation being folded so that it adopts a triangle shape, a good absorbent product for incontinent males is obtained. Examples of an absorbent core which is specially adapted for incontinent males is described in WO 91/07155A1.

In certain embodiments, the two articles are substantially identical in form and/or in absorption capacity after separation.

### BRIEF DESCRIPTION OF THE FIGURES

Embodiments of the invention will be described more closely in the following, with reference to the figures shown on the enclosed drawings.

Therein;

FIG. 1 shows an absorbent article before separation in its extended state, seen towards the liquid-permeable topsheet.

FIG. 2 shows an absorbent, non-separated article in use.

FIG. 3 shows a urine-absorbent article after separation in use.

FIG. 4 shows a urine-absorbent article after separation in use.

### DESCRIPTION OF EMBODIMENTS

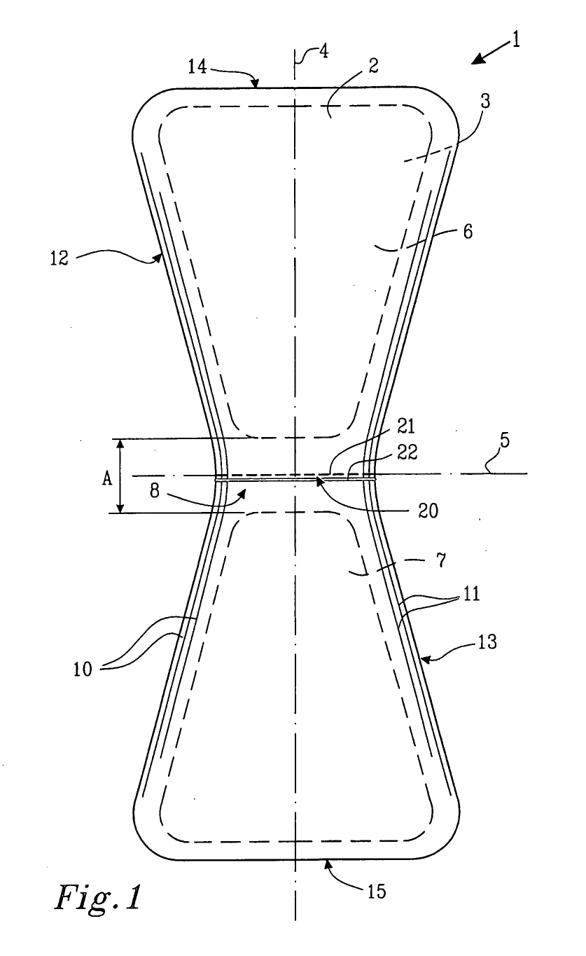

The article is described in FIG. 1 in its extended state. By the article's “extended state”, is meant that the article is shown or described in that state in which the article is stretched so that the backsheet is substantially completely planar, or as planar as the article may be when it is stretched in all corners of the article. The article exhibits a longitudinal direction, the y-direction, and a transverse direction, the x-direction.

FIG. 1 shows an absorbent article 1 intended for receiving urine and faeces, in its extended state, comprising a liquid-permeable topsheet 2, a liquid impermeable backsheet 3, a longitudinal center-line 4 and a transverse center-line 5. The article shown in FIG. 1 is intended for use on male incontinent patients. The article also has a first and a second absorbent core 6,7 placed between the liquid-permeable topsheet 2 and the liquid impermeable backsheet 3. The figure also shows the article's two longitudinal edges 12,13 as well as two transverse edges 14,15.

The absorbent cores 6,7 may be identically designed, or alternatively they may be differently designed. In the embodiment shown in FIG. 1, the first absorbent core 6 and the second absorbent core 7 have the same design, both in content and form. The absorbent cores 6,7 comprise for example a combination of cellulose fibers and superabsorbent material in a suitable mixture ratio, e.g. 50% cellulose fibers and 50% conventional superabsorbents. Other mixture ratios can also be suitable. Examples of superabsorbent materials which may be used can be found described in EP 530 517 A1, U.S. Pat. No. 5,328,935 A or U.S. Pat. No. 5,045,322. Other absorbent material than cellulose fibers and superabsorbent may naturally be used.

In certain embodiments, the absorbent cores 6,7 may be built up of more than one layers of absorbent material. Normally, the absorbent cores contain layers of porous wadding, so called reception- and distribution layers, to be able to carry away emitted liquid from the liquid-receiving topsheet 2. Each of the layers comprised in the absorbent cores 6,7 can of course contain such above-named distribution layers. A number of absorbent structures which should be able to suit the absorbent article are described further in EP 0659 541. Other examples of absorbent structures can be found in e.g. WO 93/21882. However, the invention is not only limited to these, but these should only be considered as examples.

In one embodiment, the liquid-impermeable backsheet 3 is formed from a breathable plastic film, but in another embodiment can comprise or consist of a nonwoven sheet which is covered with a liquid-blocking material, or another flexible material sheet which is able to resist liquid penetration. It is normally an advantage if the liquid-impermeable backsheet 3 exhibits breathability, i.e. allows passage or water vapour through the sheet 3. Further examples of other backsheets which may be used are described in WO98/29246A1 and WO2003/013833. The invention is not limited to these, however—rather they should be regarded as examples.

In one embodiment, the topsheet 2 is formed form a nonwoven sheet of polypropylene. Further examples of topsheets which may be used are described in EP 0 173 058 and EP 0 629 720. The invention is not limited to these, however—rather they should be regarded as examples.

The topsheet 2 and the backsheet 3 are joined to one another around the periphery of each first and second absorbent core 6,7. Between the first and the second absorbent core 6,7, the absorbent article 1 has an area 8 free from absorbent core, which is limited in its extension in the longitudinal direction of the article by the first and second absorbent cores 6,7. The distance between the first absorbent core 6 and the second absorbent core 7 is distance A (=ca. 25 cm) in the embodiment shown. The area 8 free from absorbent core reaches all the way out to the longitudinal edges 11, 12 of the absorbent article 1 in the embodiment shown.

FIG. 1 also shows longitudinal elastic threads 10,11 which run substantially along the article's two longitudinal edges 12, 13. The elastic threads 10,11 therefore run alongside the first absorbent core 6, across the article's transverse center-line 5 and alongside the second absorbent core 7. In this embodiment, there are four elastic threads 10,11. The total length of the article 1 as measured along the article's longitudinal center-line is ca. 550 mm. The length of the first and the second absorbent cores 6,7, as measured along the longitudinal center-line of the article is ca 250 mm.

In the area 8 free from absorbent core, the backsheet 3 is glued fast to the topsheet 2. This means that the first absorbent core 6 and the second absorbent core 7 do not have any contact for transport of liquid between the absorbent cores. In another embodiment, the backsheet 3 and the topsheet 2 can be welded by ultrasound, or alternatively any other joining method known in the field may be used.

In the embodiment shown in FIG. 1, a separation indication 20 is present in the area 8 free from absorbent core. The separation indication 20 is formed from a perforation 21 together with a colored dashed line 22 which clarifies the location of the perforations for a user.

Before the article 1 is used, a user can decide whether the article should be used as a urine- and faeces-receiving article, or only as urine-receiving. If the user wants to use the article both as a faeces and urine-receiving article, the article is applied as it is. Separating the article results in two substantially identical absorbent articles which may be used for urine absorption. The article in the embodiment is primarily intended for use by incontinent males, which is why a triangle-shaped absorbent core is typical.

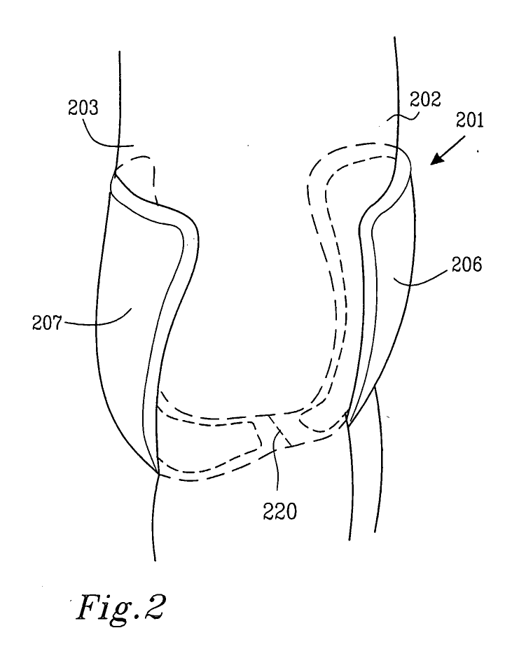

FIG. 2 shows the article 201 in use, without separation of the article 201 having occurred. Article 201 runs from the user's trunk between the legs and up towards the users backside and can thereby receive secretions of both urine and faeces. The figure shows the first absorbent core 206 placed near or in contact with the user's trunk and the second absorbent core 207 placed near or in contact with the user's backside. The separation indication 220 is placed in the middle of the user's crotch area.

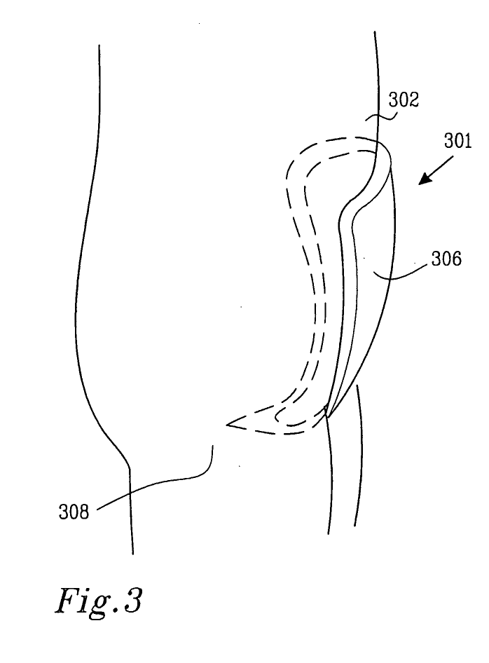

FIG. 3 shows how the absorbent article 301 looks after separation. The absorbent cores 306 lie against the user's genitalia and trunk 302, so that optimal urine absorption is obtained. The embodiment is primarily intended to be used by incontinent males. After separation, two substantially identical urine-absorbing articles are formed, one of which is shown in the figure. The newly formed absorbent article 301 spans from the trunk to the crotch area.

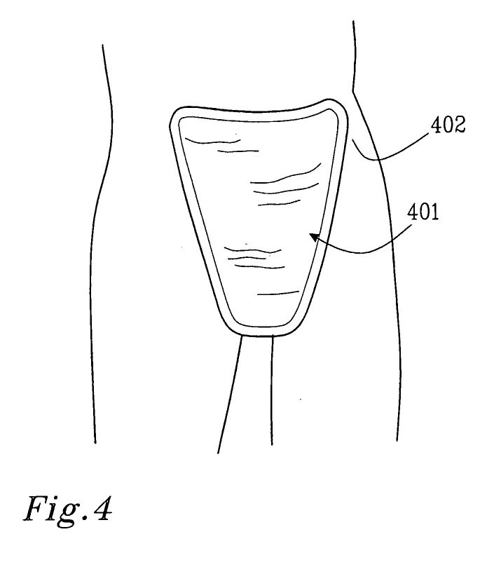

FIG. 4 shows a separated absorbent article 401 seen towards the user's trunk, i.e. from the front. The embodiment is primarily intended to be used by incontinent males.

Furthermore, the invention is not restricted to the above-mentioned illustrative embodiments, but is naturally applicable to other embodiments within the scope of the following patent claims, and equivalents thereof.

1. An absorbent article comprising a topsheet, a backsheet and at least two absorbent cores placed therebetween, wherein the article has a longitudinal direction and a transverse direction, the at least two absorbent cores are arranged at a distance from each other as seen in the longitudinal direction, and are adapted to be separable from each other, wherein, after separation, the article forms two separate articles, each of which has at least one absorbent core.

2. The absorbent article according to claim 1, wherein at least one individual separate article is primarily intended for urine absorption.

3. The absorbent article according to claim 1, wherein, prior to separation, said at least two absorbent cores are connected to each other by an area which is free from the absorbent core.

4. The absorbent article according to claim 3, wherein in said area free from the absorbent core, the topsheet and the backsheet are joined directly to each other, so that there is no direct liquid communication between said at least two absorbent cores separated from each other.

5. The absorbent article according to claim 3, wherein said area free from the absorbent core has a length seen in the longitudinal direction of 10-60 mm.

6. The absorbent article according to claim 1, wherein said article has an indication for separation, wherein upon separation along said indication, the article forms two individual separate articles.

7. The absorbent article according to claim 6, wherein the indication for separation is located in said area free from the absorbent core, between the two absorbent cores which are separated from each other.

8. The absorbent article according to claim 6, wherein said indication for separation comprises a color marking.

9. The absorbent article according to claim 6, wherein said indication for separation comprises a folding indication.

10. The absorbent article according to claim 1, wherein the two individual separate articles are substantially identical with respect to their form.

11. The absorbent article according to claim 1, wherein the two individual separate articles are substantially identical with respect to their absorption capacity.

12. The absorbent article according to claim 1, wherein the article comprises separation means.

13. The absorbent article according to claim 12, wherein said separation means is comprised of a perforation.

14. The absorbent article according to claim 1, wherein the article's length before separation is at least 400 mm.

15. The absorbent article according to claim 1, wherein after separation, said article forms two individual separate articles especially designed for male incontinence.

16. The absorbent article according to claim 3, wherein said area free from the absorbent core has a length seen in the longitudinal direction of 15-50 mm.

17. The absorbent article according to claim 3, wherein said area free from the absorbent core has a length seen in the longitudinal direction of 15-40 mm.
